(19) United States
(12) Patent Application Publication
(10) Pub. No.: US 2013/0158506 A1
(43) Pub. Date: Jun. 20, 2013

(54) CATHETER AND NEEDLE SYSTEM AND METHOD OF INSERTING A CATHETER

(71) Applicant: Tangent Medical Technologies LLC, Ann Arbor, MI (US)

(72) Inventors: Adrienne Rose Harris, Tecumseh, MI (US); Steven B. White, Ann Arbor, MI (US); Nathan Farrell, Ann Arbor, MI (US); Elyse White, Ann Arbor, MI (US); Ronald Dean Duis, Plainwell, MI (US); Henry J.H. Brown, Ann Arbor, MI (US)

(73) Assignee: Tangent Medical Technologies LLC, Ann Arbor, MI (US)

(21) Appl. No.: 13/681,149

(22) Filed: Nov. 19, 2012

Related U.S. Application Data
(63) Continuation-in-part of application No. PCT/US11/37230, filed on May 19, 2011.
(60) Provisional application No. 61/586,622, filed on Jan. 13, 2012.

(30) Foreign Application Priority Data
May 19, 2011 (US) PCT/US11/37230

(51) Int. Cl. A61B 17/34

(52) U.S. Cl. CPC A61B 17/3415
USPC 604/506; 604/164.04; 604/164.02; 604/113; 604/167.02

(57) ABSTRACT

A system and method for inserting a catheter into a patient, the system comprising a frame including a catheter hub configured to provide a first anchoring point on a patient and receive a catheter insertable in the patient at an insertion site, a stabilization hub configured to provide a second anchoring point on the patient, and a flexible tubular lateral member, extending between the catheter hub and the stabilization hub; a fluidic channel configured to fluidically communicate with the catheter and transfer fluid to the catheter; a flush fluid source configured to couple to the fluidic channel and supply flush fluid to the catheter; a housing comprising a needle mount and a flash chamber; and needle having a distal end insertable through the frame and the catheter and a proximal end coupled to the needle mount, wherein the needle is configured to provide a fluid path to the flash chamber.

### CROSS-REFERENCE TO RELATED APPLICATIONS

This application claims the benefit of U.S. Provisional Application No. 61/586,622 entitled “Catheter and Needle System and Method of Inserting a Catheter”, filed 13 Jan. 2012, which is incorporated in its entirety by this reference. This application is also a continuation-in-part of International Application Number PCT/US11/37230 entitled “Integrated Vascular Delivery System with Safety Needle”, filed 19 May 2011, which is incorporated in its entirety by this reference.

### TECHNICAL FIELD

This invention relates generally to the medical care field, and more specifically to an improved catheter and needle system and method of inserting a catheter.

### BACKGROUND

Patients undergoing medical treatment often require a form of intravenous (IV) therapy, in which a fluid is administered to the patient through a blood vessel of the patient. IV therapy is among the fastest ways to delivery fluids and medications into the body of the patient. Intravenously infused fluids, which typically include saline, drugs, blood, and/or antibiotics, are conventionally introduced to the patient through a flexible catheter positioned at any of several venous routes, such as peripheral veins and central veins.

To set up IV therapy with conventional devices and methods, a medical practitioner (e.g., nurse, physician, or other caregiver) positions the catheter over the selected blood vessel and uses a needle within the catheter to pierce the skin, enter the blood vessel and allow insertion of the distal end of the catheter over the needle into the blood vessel. Typically, when the needle and catheter are properly placed, blood will flow through the catheter and extension tubing (external tubing) that is connected to the catheter. The caregiver connects the catheter to a fluid supply through the extension tubing and other external tubing. After the catheter is inserted and fluidically coupled to the fluid supply, fluid is administered to the patient through the tubing and catheter.

However, the medical practitioner may encounter some difficulties in setting up IV therapy, which may result in complications for the patient. For example, if the patient does not have adequate blood flow, upon catheter entry into the blood vessel, the extension tubing may not completely fill with blood flowing out from the patient. As a result, when the medical practitioner flushes the catheter by inducing flow of a fluid into the patient, there is potential for a trapped air bubble to be infused into the patient, which may develop into a dangerous and possibly fatal air embolism or other complications. As another example, the needle must be correctly positioned within the blood vessel to enable the proper placement of catheter for IV therapy, but this can be difficult to determine before the catheter is inserted into the blood vessel.

Thus, there is a need in the medical care field to create an improved catheter and needle system and method of inserting a catheter.

### BRIEF DESCRIPTION OF THE FIGURES

FIGS. 1A and 1B are perspective and top view schematics, respectively, of the catheter and needle system of a preferred embodiment;

FIGS. 2A and 2B are perspective and side view schematics, respectively, of the catheter system of a preferred embodiment;

FIG. 2C is a schematic of an embodiment of a catheter hub and needle;

FIG. 2D is a schematic of an embodiment of a safety needle system;

FIGS. 3A and 3B are a detailed cross-section schematic of the catheter and needle tips and side view schematic of the catheter and needle system, respectively, of a preferred embodiment;

FIGS. 4A-4D show alternative embodiments of the fluidic channel and extension tubing;

FIG. 5 is a flow chart of an embodiment of a method of inserting a catheter; and

FIGS. 6A-6H are schematics of the method of inserting a catheter of a preferred embodiment.

### DESCRIPTION OF THE PREFERRED EMBODIMENTS

The following description of preferred embodiments of the invention is not intended to limit the invention to these preferred embodiments, but rather to enable any person skilled in the art to make and use this invention.

### 1. System for Inserting a Catheter

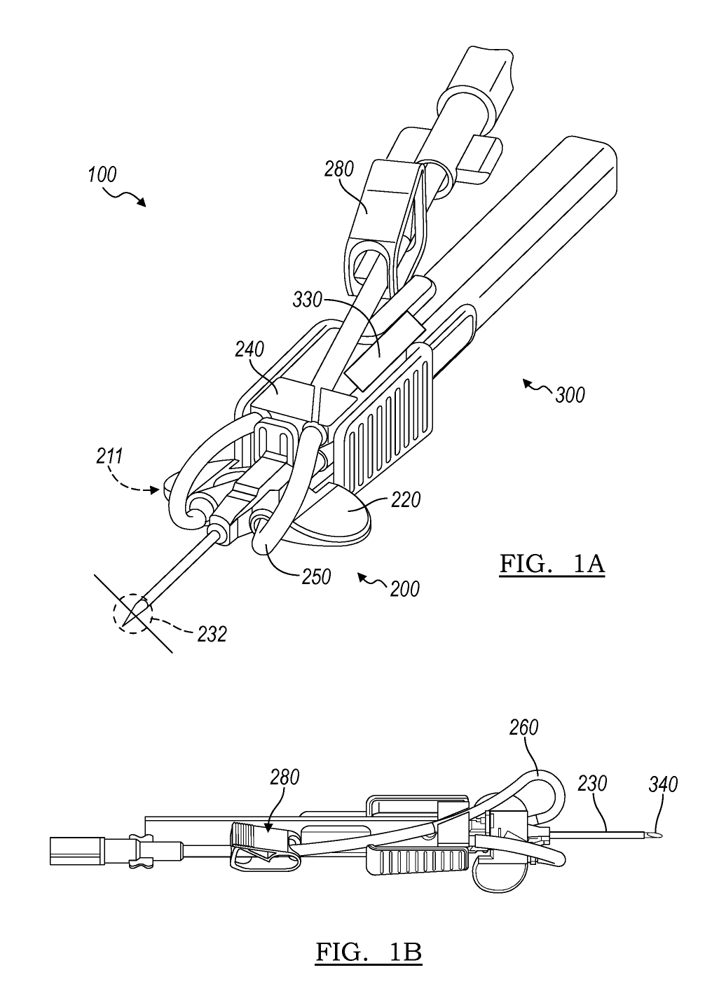

As shown in FIGS. 1A and 1B, the system 100 for inserting a catheter of a first preferred embodiment comprises an integrated vascular delivery system 200 and a safety needle system 300. In an alternative embodiment, the system focuses only on the integrated vascular delivery system 200 and, in yet another alternative embodiment, the system focuses only on the safety needle system 300.

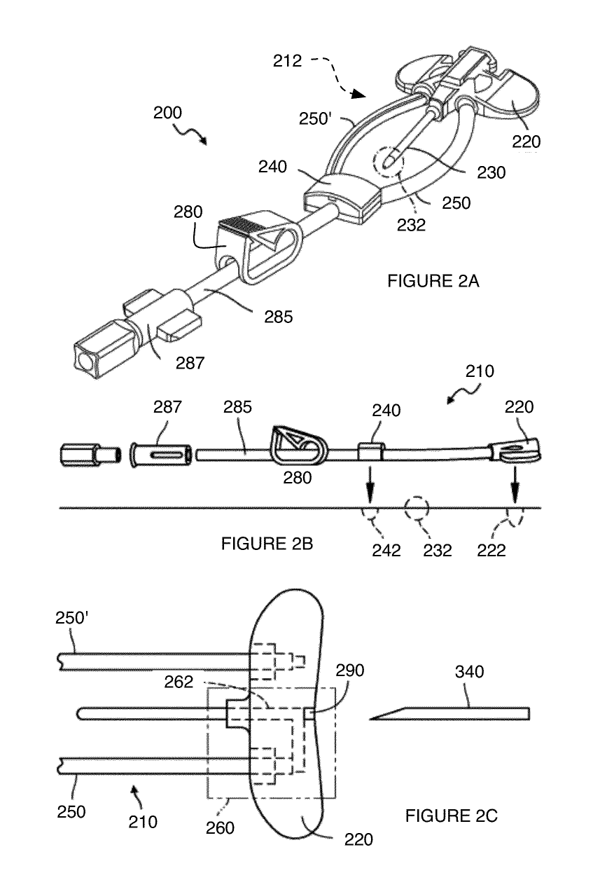

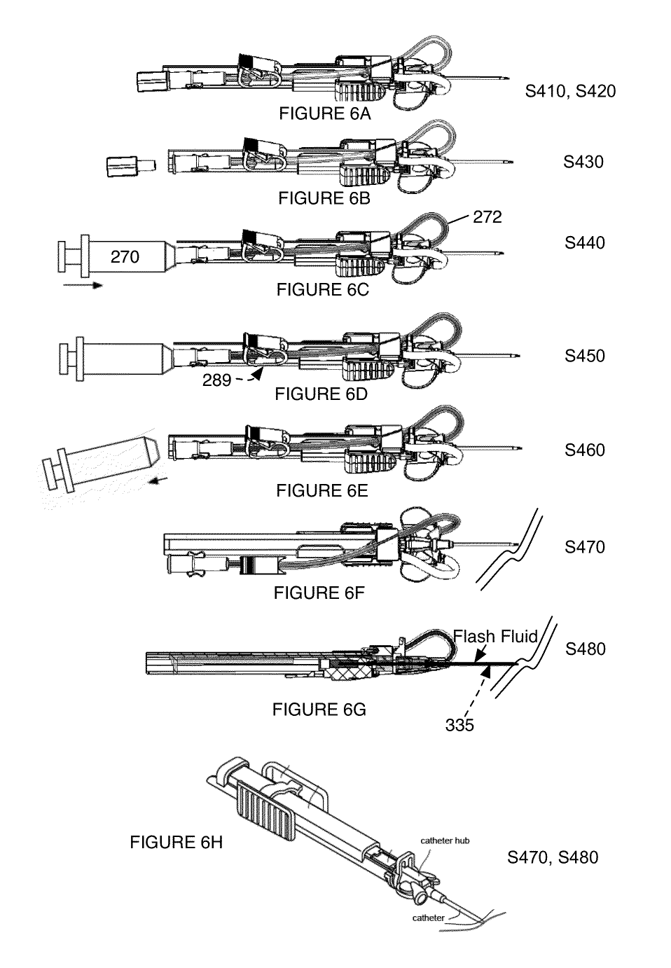

As shown in FIGS. 2A and 6D, the integrated vascular delivery system 200 preferably comprises: a frame 210 comprising a catheter hub 220, a stabilization hub 240, and a flexible tubular lateral member 250 extending between the catheter hub 220 and the stabilization hub 240; and a fluidic channel 260 configured to fluidically communicate with the catheter 230 and transfer a fluid to the catheter 230. The integrated vascular delivery system may further comprise a flush fluid source 270 configured to couple to the fluidic channel 260 and supply a flush fluid 272 to the catheter 230. The catheter hub 220 is preferably configured to provide a first anchoring point 222 on a patient and receive a catheter 230 insertable in the patient at an insertion site 232, and the stabilization hub is preferably configured to provide a second anchoring point 242 on the patient. The frame of the integrated vascular delivery system preferably operates in a folded configuration 211 that facilitates insertion of the needle and catheter into the patient, and in an unfolded configuration 212 in which the first and second anchoring points 222, 242 are distributed around the insertion site 232 to anchor the frame 210 to the patient, thereby stabilizing the catheter 230. For instance, in a preferred embodiment the first and second anchoring points 222, 242 are proximal and distal to the site, respectively, or on opposite lateral sides of insertion site 232, such that the frame at least partially surrounds the catheter 230 and the insertion site 232. The integrated vascular delivery system 200 preferably further includes a tubing clamp 280 (or is configured to receive a tubing clamp) that selectively restricts flow through an extension tubing 285, but may additionally and/or alternatively include or be configured to receive a valve, plug, or any suitable means for selectively restricting or preventing flow through the external tubing. The integrated vascular delivery system 200 is preferably the system described in International Application Number PCT/US11/37230 entitled “Integrated Vascular Delivery System with Safety Needle”, which is incorporated in its entirety by this reference. However, the integrated vascular delivery system 200 may include any suitable closed catheter system, or any other suitable catheter system.

As shown in FIGS. 1A and 1B, the system 100 for inserting a catheter of a preferred embodiment comprises an integrated vascular delivery system 200 with safety needle system 300. As shown in FIGS. 1A and 2D and 3B, the safety needle system 300 preferably comprises: a housing 310 comprising a needle mount 320 and flash chamber 330, and a needle 340 having a distal end insertable through the frame 210 and the catheter 230, and a proximal end coupled to the needle mount 320, wherein the needle is configured to provide a fluid path to the flash chamber 330. Preferably, the safety needle system 300 further comprises a sheath 350 configured to telescopically engage with the housing, and a slider 360 configured to engage with at least one of the sheath and the housing. The safety needle system is preferably that described in International Application Number PCT/US11/37230 entitled “Integrated Vascular Delivery System with Safety Needle”, which is incorporated in its entirety by this reference.

### 1.1 Integrated Vascular Delivery System

The integrated vascular delivery system 200 of the system 100 for inserting a catheter preferably comprises a frame 210, which functions to stabilize the integrated vascular delivery system 200 and a catheter 230 in relation to a patient. As shown in FIGS. 2A and 2B, the frame 210 preferably comprises a catheter hub 220 configured to provide a first anchoring point 222 on a patient and configured to receive a catheter 230 insertable in the patient at an insertion site 232, a stabilization hub 240 configured to provide a second anchoring point 242 on the patient, and a flexible tubular lateral member 250 defining a lumen, extending between the catheter hub and the stabilization hub. Preferably, the frame 210 comprises two lateral members, including the flexible tubular lateral member 250, thus forming a perimeter about the distal end of the catheter 230 and the insertion site 232; however, the frame may alternatively comprise any suitable number of hubs and any suitable number of lateral members, such that the frame forms an enclosed or partial, non-enclosed perimeter of any suitable shape and size, with any number of anchoring points, around an insertion site 232. The frame 210 preferably allows visualization of the insertion site 232, such as by leaving an open uncovered area about the catheter, although alternatively, the frame 210 may include a cover that is transparent, translucent, opaque, or any suitable kind of material, that extends to cover the insertion site 232 and/or catheter 230.

The frame 210 preferably comprises a catheter hub 220, which functions to provide a point of coupling to the safety needle system 300 and to stabilize the catheter 230 with respect to a patient. The catheter hub is preferably configured to provide a first anchoring point 222 on a patient and configured to receive a catheter 230, which may be embedded in the catheter hub and integrally part of the integrated vascular delivery system 200, or alternatively may be a separate catheter 230 that is coupled to the catheter hub 220. The catheter hub 220 preferably includes a channel, concentrically aligned with the catheter, that may receive a needle 340 used during insertion of the catheter 230 into the patient. In one embodiment, the catheter hub may include a sensor that is configured to measure a biometric parameter, such as temperature, blood pressure, or pulse rate of a patient. The sensor may additionally and/or alternatively sense any suitable parameter such as one pertaining to the fluid passing through the catheter, such as pH or flow rate.

The frame 210 also comprises a stabilization hub 240, which also functions to stabilize the integrated vascular delivery system 200 with respect to a patient. Preferably, the stabilization hub 240 is configured to provide a second anchoring point 242 on the patient. In one embodiment, as shown in FIGS. 1A and 2A, the stabilization hub is configured to couple to a sheath 350, and further configured to couple to an extension tubing 285 that functions to transfer a fluid or a flush fluid 272 to the catheter 230 by the fluidic channel 260. The stabilization hub 240 may also include a sensor that is configured to measure a biometric parameter, such as temperature, blood pressure, or pulse rate of a patient. The sensor may additionally and/or alternatively sense any suitable parameter such as one pertaining to the fluid passing through the catheter, such as pH or flow rate.

The catheter hub 220 and/or stabilization hub 240 may have a relatively wide and this profile, which may help distribute forces over a greater area on the skin and decreases the chances of the patient developing skin irritations, sores, and other degradations. The thin profile may help decrease the risk of the catheter and/or stabilization hubs 220, 240 catching or snagging on equipment or any other objects in close proximity to a patient being treated by the system 100, as interactions with such equipment or objects could cause the catheter to move within the vein and cause complications such as catheter dislodgement, infiltration, and phlebitis. However, the catheter and stabilization hubs 220, 240 may have any suitable shape, and the catheter hub 220 may have a different shape from the stabilization hub 240, as shown in FIG. 1A, in an embodiment where the catheter and stabilization hubs 220, 240 have different coupling configurations. The catheter and stabilization hubs 220, 240 may include a rigid or semi-rigid plastic or other suitable material, and/or softer material. For example, one of both hubs may include a rigid core overmolded with a softer material, such as silicone.

As shown in FIG. 2A, the integrated vascular delivery system 200 may further include an extension tubing 285 with a fluid supply adapter 287 and a flow restriction mechanism, which function to transfer fluids in a controlled manner to the catheter 230. The extension tubing 285, which provides stress relief if the system 100 is jostled (such as from patient movement or caregiver manipulations), is preferably made of flexible tubing such as polymer tubing, but may alternatively be a passageway made of any other suitable material. The extension tubing 285 is preferably long enough to provide stress relief if needed, but short enough to reduce the chances of the extension tubing 285 catching or snagging on nearby obstacles. In another variation, the extension tubing 285 may be coiled like a spring to provide stress relief. The length of the extension tubing may alternatively be any suitable length, and may depend on the specific application of the system. Other dimensions of the extension tubing 285, such as outer diameter and inner diameter, may also depend on the specific application of the system 100. The fluid supply adapter 287 preferably includes a connector that attaches the extension tubing 285 to a fluid supply (e.g. pole-mounted IV bag, syringe, flush fluid source, or pump that supplies fluid through tubing). The connector may be a standard female luer lock connector (FIGS. 2A and 2B), or Y-connector that commonly interfaces with conventional IV bags. Alternatively, the connector may be any suitable male or female connector that is adapted to interface with a fluid supply. Furthermore, the luer lock connector or other fluid supply adapter 287 maybe coupled directly to the catheter hub 220 and/or stabilization hub 240, rather than to an extension tubing 285. The flow restriction mechanism is preferably a tubing clamp 280, as shown in FIGS. 2A and 2B, but alternatively can be a valve (e.g. stopcock) or any other mechanism for restricting fluid flow through a channel. In one embodiment, flow restriction mechanism is a tubing clamp 280, which functions to reversibly restrict fluid flow through the extension tubing 285 at a restriction point 289, thus providing transfer of fluids in a controlled manner. Preferably, the tubing clamp 280 is coupled to the extension tubing 285, which is fluidically coupled to the fluidic channel 260. Preferably, when the tubing clamp 280 is used, fluid flow is prevented from passing the restriction point 289 of extension tubing, and results in a lack of a pressure differential across the fluidic channel 260.

The frame 210 preferably also comprises a flexible tubular lateral member 250, which functions to provide a passage for a portion of the fluidic channel 260, and to provide structural stability to the frame 210 by stabilizing the catheter hub 220 relative to the stabilization hub 240. As shown in FIGS. 2A and 2B, the frame preferably includes two lateral members 250, 250′, comprising a flexible tubular lateral member 250, that, with the catheter and stabilization hubs 220, 240, form a perimeter about the catheter 230. The configuration of the two lateral members 250, 250′ preferably results in the formation of an approximately ellipsoid perimeter about the catheter 230, but alternatively, the two lateral members may be configured in a parallel (i.e. resulting in the formation of an approximately rectangular perimeter), crossed, non-parallel, or any other suitable configuration. Each lateral member 250, 250′ may be flexible, such as to allow the catheter and stabilization hubs 220, 240 to move relative to one another with a significant number of degrees of freedom, including displacement in a compression direction (and subsequent displacement in a tension direction) along the axis of the catheter 230, displacement in directions along axes not parallel (e.g. perpendicular) to the axis of the catheter 230, twisting along axes parallel the axis of the catheter 230, and bending along axes not parallel (e.g. perpendicular) to the axis of the catheter. The second lateral member 250′ may be tubular or solid (e.g. a dummy lateral member), such that it provides structural stability but does not provide a passage. Alternatively, the frame may include only a partial perimeter about the catheter 230, such as with one lateral member instead of two.

The integrated vascular delivery system 200 also comprises a fluidic channel 260, which functions to deliver a fluid from a fluid supply to the catheter 230, and in some embodiments, deliver a fluid to and from the catheter 230, such as in transferring fluid removed from the patient through the catheter 230 to a reservoir. The fluid is either a fluid intended to be administered to a patient (e.g. fluid comprising medication), or a flush fluid, as described below. As shown in FIGS. 2C and 6A-6D, at least a portion of the fluidic channel 260 may be fixed within at least one of the catheter and stabilization hubs 220, 240, and/or within the flexible tubular lateral member 250. As shown in FIGS. 2A and 2B, at least a portion of the fluidic channel 260 may be additionally and/or alternatively be external to the catheter and stabilization hubs 220, 240 and flexible tubular lateral member 250. For instance, a least a portion of the fluidic channel 260 may be molded to an external surface of the catheter hub 220, the stabilization hub 240, and/or flexible tubular lateral member 250. The fluidic channel 260 preferably includes a turnabout portion 262 in which a fluid flows in a direction different from that within the catheter 230. In particular, the turnabout portion 262 preferably angularly displaces a fluid flow direction by approximately 180 degrees, but alternatively angularly displaces a fluid flow direction by an amount less than or greater than 180 degrees. The turnabout portion 262 of the fluidic channel 260 may be fixed or embedded within the catheter hub 220 and/or the stabilization hub 240. In one exemplary application of the system 100, the catheter 230 is inserted in the patient, such that its penetrating end points proximally towards the heart of the patient, and the turnabout portion 262 of the fluidic channel 260 allows a stand supporting the IV bag or other fluid supply to be kept near the head of a bed, or otherwise proximal to the insertion site 232 as is typically practiced in patient treatment settings. The internalized fluid flow turn in the turnabout portion 262 of the fluidic channel 260 reduces the number of external structures that can get caught or snagged on nearby obstacles and consequently disturb the catheter and IV setup. Another effect of the turnabout portion 262 is that if an extension tubing 285 in the IV setup is pulled or caught, the turnabout portion 262 may enable the frame 210 to stabilize the catheter 230 more effectively by causing the catheter 230 to be pulled further into the patient. For example, in a common catheter placement in which the catheter 230 is placed on the forearm with its distal end pointing proximally toward the elbow of the patient, if the extension tubing 285 is accidentally pulled posteriorly towards the patient, the tubing will in turn pull the turnabout portion 262 of the fluidic channel 250 and the catheter hub 220 toward the patient, thereby pulling the catheter 230 further into the blood vessel of the patient rather than displacing the catheter 230 from the insertion site 232.

The integrated vascular delivery system 200 may also further comprise a flush fluid source 270, which functions to supply a flush fluid for removing gas bubbles that may be trapped along the fluidic channel 260. The flush fluid source 270 is preferably configured to supply a flush fluid 272 through the fluidic channel 260 and/or the catheter 230, thus allowing the fluidic channel 260 and/or catheter 230 to be completely filled with the flush fluid 272 prior to insertion of the catheter 230 into a patient. Preferably, as shown in FIG. 6D, the flush fluid source 270 is a syringe that is configured to be manually pumped to supply the flush fluid 272 to the fluidic channel 260 and/or the catheter 272. Alternatively, the flush fluid source 270 comprises a manual or automated mechanical pump (e.g. syringe pump or infusion pump), or other suitable mechanism for supplying a flush fluid 272. In an embodiment, the flush fluid source 270 may provide a specific volume of the flush fluid 272 to flush the integrated vascular delivery system 200, and/or provide the flush fluid at a specific temperature (e.g. 37 degrees Celsius) using a temperature regulator, thermocontrol, heat monitor, or other appropriate device. Preferably, the flush fluid source 270 is coupled to the fluidic channel 260 using a luer connector, which may or may not be coupled to an extension tubing 285, as shown in FIGS. 2A and 6A-6D; however, in alternative embodiments any suitable connector may be used to couple the flush fluid source 270 to the fluidic channel 260. Preferably, the flush fluid 272 is saline (e.g. 0.9% normal pH sodium chloride saline); however, the flush fluid may alternatively be any sterile fluid or other suitable flush fluid, such as a medication-containing fluid intended to be transferred to a patient by the catheter.

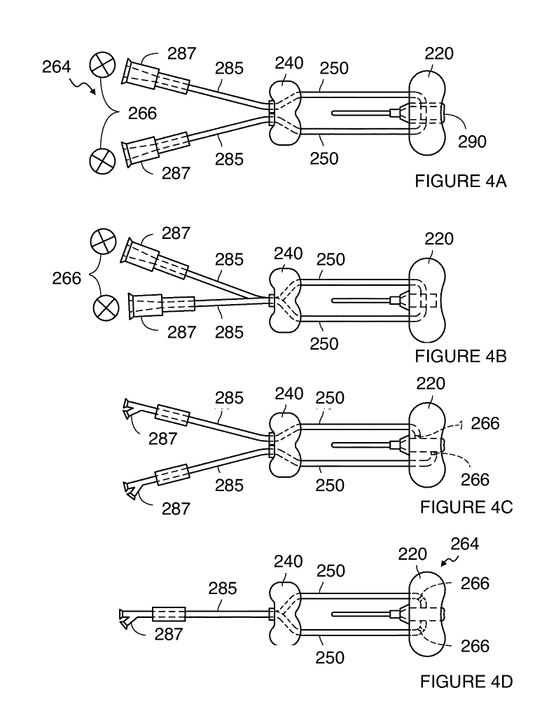

In a preferred embodiment, the integrated vascular delivery system 200 includes a single fluidic channel 260 configured to transfer one fluid (e.g. flush fluid or other fluid) at a time through the fluidic channel 260, as shown in FIG. 2A-2C. In the preferred embodiment, the single fluidic channel is configured to be coupled to the flush fluid source 270 (or other fluid supply) by an extension tubing 285 and fluid supply adapter 287 at one end, configured to pass through the stabilization hub 240 and the flexible tubular lateral member 250, comprise a turnabout portion 262 that is fixed within the catheter hub 220, and couple to a catheter 230 at another end. However, in alternative embodiments system 100 may include one, two, or any suitable number of fluidic channels, each configured to couple (directly or indirectly) to a flush fluid source 270 supplying a flush fluid 272. For instance, in one alternative embodiment a second fluidic channel 260′ may pass through a second lateral member 250′. The second fluidic channel 260′ preferably receives a second fluid, which may be the same or different from the fluid supplied to the first fluidic channel 260. As shown in FIG. 4A-4C, the system may further include a second extension tubing 285′ configured to supply a second fluid to the frame and catheter, and configured to couple to a flush fluid source 270. However, as shown in FIG. 4D, the system may include only one extension tubing 285 that is configured to couple to a flush fluid source 270, and configured to supply fluid to one or multiple fluidic channels. The fluidic channels may have separate inlets on the stabilization hub 240 (FIGS. 4A and 4C), or may share the same inlet on the stabilization hub 240 in which flow may be regulated with valves or other fluid control means (FIGS. 4B and 4D). In one variation, a first and second fluidic channel preferably each fluidically communicate with the same catheter 230 in the catheter hub 220, coupled to the catheter 230 at the same point (FIGS. 4A and 4B) or different points (FIG. 4C) along the length of the catheter 230 or channel. In this variation, the system preferably includes a flow control system 264 that selectively restricts flow of one or both of the fluids to the catheter and therefore to the patient. The flow control system 264 may include one or more valves 266, such as at the extension tubes (FIGS. 4A and 4B), at the junction between the fluidic channel 260 and the catheter 230 (FIGS. 4C and 4D) or any suitable location. The flow control system may additionally and/or alternatively use pressure drops, vents, or any suitable technique for controlling fluid flow among the fluidic channels and catheter 230. The flow control system may also be present in an embodiment that includes only one fluidic channel 260. In another variation, the first and second fluidic channels preferably fluidically communicate with a catheter 230 with dual lumens, such that one catheter lumen is coupled to the first fluidic channel and another catheter lumen is coupled to a second fluidic channel. In yet another variation, the first and second fluidic channels fluidically communicate with separate catheters. Additional variations expand on these variations with three or more fluidic channels.

While intended to be used with an embodiment of the safety needle system 300 described below, the integrated vascular delivery system 200 may be used with another needle system.

### 1.2 Safety Needle System

The safety needle system 300 of the system 100 for inserting a catheter preferably comprises a housing 310 and a needle 340. Preferably, the safety needle system 300 further comprises a sheath 350 configured to telescopically engage with the housing, and a slider 360 configured to engage with at least one of the sheath and the housing.

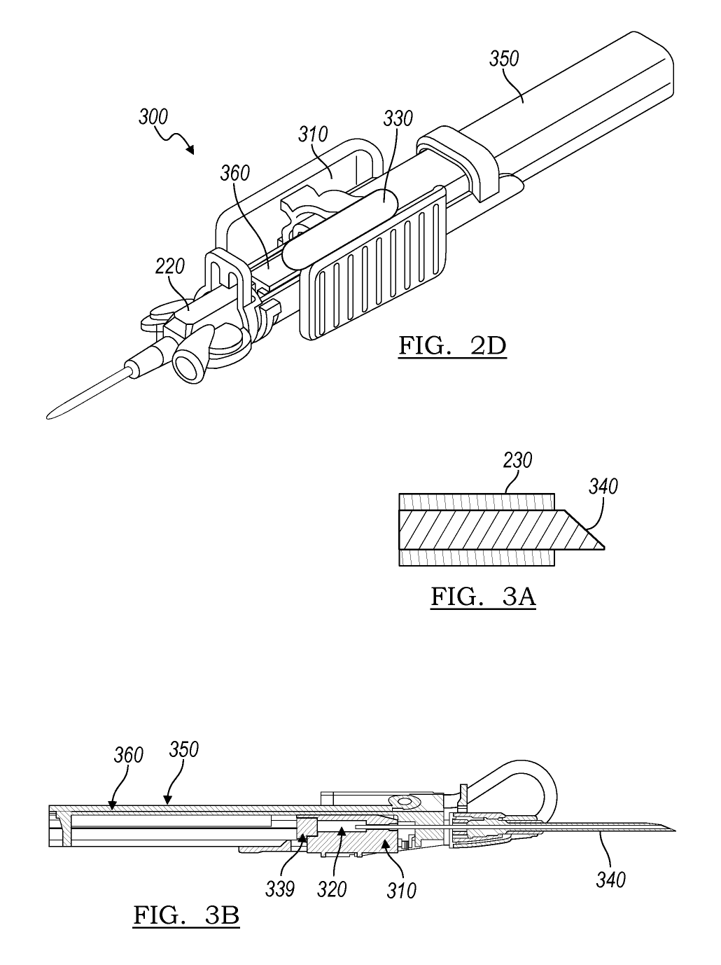

The housing 310 preferably comprises a needle mount 320 and a flash chamber 330, and functions to couple to and support a needle 340 and indicate that a blood vessel has been penetrated by the needle 340. The housing 310 also functions to support the sheath 350 and the slider 360 and/or to provide a user interface. As shown in FIGS. 1A and 3B, the needle mount 320 is configured to be coupled to a needle 340. The needle mount 320 is preferably on a distal end of the housing 310 and axially centered on the housing 310, but may alternatively be on any suitable portion of the housing 310. The needle 340 may be molded into the needle mount 320 such that the distal end of the needle 340 extends out of the distal end of the housing 310, but the needle may alternatively be coupled to the needle mount with a snap fit, friction fit, threads, epoxy, or in any suitable manner. The housing 310 is preferably the housing described in International Application number PCT/US11/37230 entitled “Integrated Vascular Delivery System with Safety Needle” (which is incorporated in its entirety by this reference), further comprising a flash chamber 330, as described below.

The flash chamber 330 of the housing 310 functions to provide an indication that a blood vessel in a patient has been penetrated by the needle 340 and/or catheter 230. The flash chamber 330 is preferably a reservoir embedded within the housing 310 and connected to a fluid path 335 that is configured to provide fluidic access by the needle 340 to a blood vessel being penetrated (FIG. 6H). Preferably, the fluid path 335 is separated from the fluidic channel 260, but alternatively the fluid path 335 is not separated from the fluidic channel 260. Preferably, the flash chamber is located near the proximal end of the needle mount, such that the fluid path 335 is configured to fluidically couple to the flash chamber 330 at the distal end of the needle 340, traverse the length of the needle 340, and provide access to blood vessel being monitored. Alternatively, the flash chamber 330 is not embedded within the housing 310, but is rather a peripheral chamber coupled to the exterior of the housing 310. Alternative configurations of the flash chamber 330 relative to the housing comprise all variations where the flash chamber 330 is configured to fluidically couple to the blood vessel being penetrated and provide indication that the blood vessel has been penetrated. The flash chamber 330 preferably includes a vent 339 that exposes the flash chamber 330 to approximately atmospheric pressure, such as by defining a perforation (e.g., slit or hole) or including a gas permeable membrane or other material. The vent is preferably at a proximal end of the flash chamber 330 (in the embodiment where the flash chamber is a reservoir) relative to the patient, but may alternatively be in any suitable location that provides any suitable pressure differential between the flash chamber 330 and the distal end of the needle 340 when the needle is inserted into the blood vessel. The vent thus provides pressure relief, such that the flash chamber can fill with flash fluid (e.g. blood) when the blood vessel is penetrated. In some alternative embodiments, the safety needle system 300 may additionally and/or alternatively include a sensor (e.g., chemical sensor, impedance sensor) that indicates visually and/or non-visually to the user when flash fluid has entered the flash chamber 330 and/or the needle 340 has entered the blood vessel.

The needle 340 of the safety needle system 300 functions to penetrate the blood vessel of a patient, and provide a fluid path 335 to the flash chamber 330. As shown in FIG. 4B, the needle 340 preferably has a lumen, but alternatively, the needle 340 may be a solid needle that has no lumen. In an embodiment of the safety needle system 300 where the needle 340 has a lumen, lumen of the needle is preferably continuous and uninterrupted (e.g., no “notch” or cutaway portion along the needle length) and provides a direct fluid path 335 to the flash chamber. This fluid path enables an amount of blood or other flash fluid to travel from the needle 340 to the flash chamber upon needle placement within a blood vessel. In alternative embodiments with a solid needle having no lumen, the needle 340 comprises a notch or groove traversing the length of the needle 340, which forms a fluid path 335 coupled to the flash chamber 330 when the needle is engaged within the catheter 230. In these alternative embodiments, the fluid path may pass through a coupler between the catheter hub 220 and the housing 310, such that the fluid path is continuous between the blood vessel and the flash chamber 330.

The needle 340 is preferably configured to form a tight fit with the catheter, such that no fluid (e.g. flush fluid, flash fluid, or other fluid) may pass through an annular region defined between the catheter 230 and the needle 340, aside from passing through the fluid path 335. As shown in FIG. 4A, prior to insertion, the catheter 230 and needle 340 are preferably coupled such that the needle 340 is telescopically engaged within the catheter 230 and ready to pierce the skin of the patient. Near the distal end of the catheter 230, the catheter 230 and needle 340 are preferably tightly fit enough so as to form a fluidic seal that prevents fluid flow from the blood vessel into the annular region between the catheter 230 and the needle 340 (e.g., friction fit or tighter) during insertion of the needle into the blood vessel. However, the fluidic seal may additionally and/or alternatively be formed by a gasket or other sealing mechanism. When the tubing clamp 280 is engaged and restricts flow through the fluidic channel 260 at a particular clamp location, a fixed volume in the fluidic channel is preferably defined between the clamp location and the fluidic seal formed by the catheter 230 and needle 340. Other respective portions of the integrated vascular delivery system 200 and the safety needle system 300 may also be engaged to stabilize the coupling of catheter 230 and the needle 340, overall integrated vascular delivery system 200 and safety needle system 300, and/or help maintain the integrated vascular delivery system 200 in the folded configuration. Furthermore, in embodiments of the safety needle system 300 comprising a sensor that indicates flash fluid has entered the flash chamber, the sensor may be integrated with the needle 340 (e.g. along the fluid path).

In some embodiments, the safety needle system 300 further comprises a sheath 350 configured to telescopically engage with the housing, and a slider 360 configured to engage with at least one of the sheath and the housing. At least a portion of each of the housing 310, sheath 350, and slider 360 is preferably translucent (or transparent) to allow visualization of flash fluid within the flash chamber 330. Alternatively, a sensor configured to indicate that flash has entered the fluid path 335 coupled to the flash chamber 330 may be integrated with housing 310, sheath 350, slider 360, and/or needle 340. The safety needle system 300 is preferably that described in International Application number PCT/US11/37230 entitled “Integrated Vascular Delivery System with Safety Needle” but further providing visualization of flash through the housing 310, sheath 350, and/or slider 360, and/or comprising a sensor that is configured to indicate that flash fluid has entered the fluid path 335 coupled to the flash chamber 330. However, in other embodiments, the safety needle system 300 may be any suitable system that includes a flash chamber 330 (possibly comprising a vent) and a needle 340 forming a fluid path 335 in fluidic communication with the flash chamber 330.

The fluidic channel 260, catheter 230, fluid path 335 and/or other suitable part of the integrated vascular delivery system 200 or safety needle system 300 may include a sealing passageway (e.g., septum) through which the needle may enter to telescopically engage with the catheter. The sealing passageway helps to prevent escape of fluid from the fluidic channel 260, catheter 230, fluid path 335, and/or other suitable part of the integrated vascular delivery system 200 or safety needle system 300, for example, in sealing around the circumference of the needle 340 when the needle is inserted. In the example, the sealing passageway preferably additionally seals the point of needle entry after the needle 340 is removed (e.g., after catheter placement), thereby enabling leak-free, safe separation of the flash chamber 330 of the needle housing and the fluidic channel 260. For example, the passageway can be an elastomeric septum with a weakened portion that seals around the circumference of the needle while permitting entry of the needle into the fluidic channel. Other variations of the sealing passageway are described in International Application Number PCT/US11/37230 entitled “Integrated Vascular Delivery System with Safety Needle”, although the sealing passageway may be any suitable kind of septum 290 or other structure.

As described, the integrated vascular delivery system 200 and safety needle system 300 are preferably used to establish access to a blood vessel of a patient, such as one undergoing intravenous (IV) therapy. In particular, the integrated vascular delivery system 200 and safety needle system 300 are preferably used to establish access to a peripheral vein or artery such as on the arm, hand, or leg, or for central venous access on the neck, chest, abdomen, or any suitable IV location. Alternatively, the system may be used to establish catheter-based access to any suitable location, such as transfer of cerebrospinal fluid.

While intended to be used with an embodiment of the integrated vascular delivery system 200 described above, the safety needle system 300 may be used with another catheter system.

### 2. Method of Inserting a Catheter

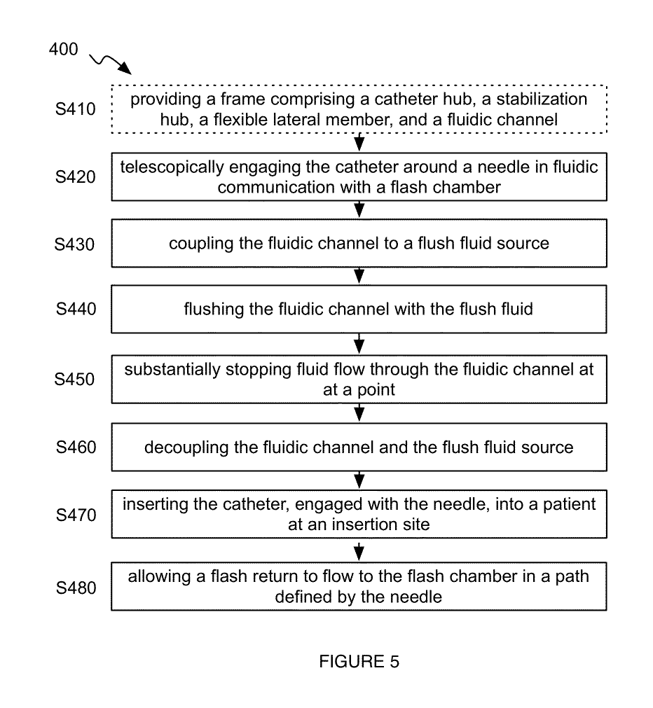

As shown in FIGS. 5 and 6A-6H, in a preferred embodiment, the method 400 of inserting a catheter into a patient comprises: providing a frame S410 comprising a catheter hub, a stabilization hub, a flexible lateral member, and a fluidic channel; telescopically engaging the catheter around a needle in fluidic communication with a flash chamber S420; coupling the fluidic channel to a flush fluid source supplying a flush fluid S430; flushing the fluidic channel with the flush fluid S440, thereby displacing any gas volume within the fluidic channel; substantially stopping fluid flow through the fluidic channel at a point, thereby defining a flush fluid volume between the point and the distal end of the catheter S450 that is maintained within the fluidic channel; decoupling the fluidic channel and the flush fluid source S460; inserting the catheter, engaged with the needle, into the patient at an insertion site S470, and allowing a flash fluid to flow through the continuous lumen of the needle to the flash chamber in a path defined by the needle S480. After catheter insertion, the fluidic channel may be coupled to a therapeutic fluid source (e.g. fluid comprising saline or fluid comprising medication) and the restriction upon the fluidic channel may be released. Preferably, the flush fluid is saline and the flash return is blood from the patient. However, the flush fluid may be any sterile fluid or other suitable flush fluid, and the flash return may be any suitable fluid and may depend on the particular application of the system (e.g., inserted in non-vascular structures). The method 400 is preferably used to both (1) preflush the fluidic channel of the catheter system (i.e., flush prior to catheter insertion into the patient) to reduce the possibility of infusion of gas (e.g., air) in the fluidic channel into the patient, and (2) enable the user to verify proper placement of the needle within the blood vessel or other targeted conduit by viewing flash return in the flash chamber.

Providing a frame S410 functions to provide a means for supplying a fluid to a patient, and mechanism for indicating that a blood vessel has been penetrated. The frame preferably comprises a catheter hub coupled to a catheter, a stabilization hub, a flexible lateral member defining a lumen and extending between the catheter hub and the stabilization hub, and a fluidic channel that fluidically communicates with the catheter and passes through the lumen of the flexible lateral member. The frame is preferably that described above, and the method 400 for inserting a catheter is preferably used with the system 100 described above, comprising an integrated vascular delivery system 200 and safety needle system 300. However, the method 400 may be used with any suitable system having a catheter, a flash chamber (in some embodiments comprising a vent), and a needle providing a fluid path in fluidic communication with the flash chamber.

Telescopically engaging the catheter around a needle in fluidic communication with a flash chamber S420 functions to facilitate penetration of the catheter into the patient, and to provide blood access to a flash chamber by a fluid path. As shown in FIG. 6A, telescopically engaging the catheter around a needle in fluidic communication with a flash chamber S420 may include coupling the integrated vascular delivery system 200 to the needle 340 of the safety needle system 300 such that the needle 340 is telescopically engaged within the catheter (this may occur during assembly by the manufacturer, and/or may be performed by the user) and/or removing a vent plug or other cover or connector coupled to the fluidic channel (FIG. 6B). The catheter and needle are preferably engaged in a fit tight enough so as to form a fluidic seal that prevents fluid flow from the blood vessel into the annular space between the catheter and the needle (e.g., friction fit or tighter). However, a fluidic seal may be formed in any suitable manner.

Coupling the fluidic channel to a flush fluid source supplying a flush fluid S430 functions to prepare the system 100 for preflushing, thus preventing a gas bubble from being trapped within the system 100 prior to insertion of the catheter 230 into a patient. As shown in FIG. 6C, coupling the fluidic channel to a flush fluid source supplying a flush fluid S430 preferably includes connecting the fluidic channel to a syringe containing saline or other suitable flush fluid (e.g. 0.9% normal pH sodium chloride saline). For example, in a preferred embodiment the fluidic channel includes a connector such as a luer connector. However, any suitable connector may be used. In other variations, the flush fluid source may be a manual or automated mechanical pump, or other suitable mechanism for supplying a flush fluid. Furthermore, coupling the fluidic channel to a flush fluid source may include preparing the flush fluid such as measuring a particular volume of fluid or preparing the flush fluid to a particular temperature (e.g. body temperature), for instance, by using embodiments of the system 100 described above.

Flushing the fluidic channel with the flush fluid S440 functions to displace gas (e.g., air) from the fluidic channel and/or catheter prior to insertion of the catheter into the patient. In an embodiment, flushing the fluidic channel with the flush fluid S440 may further comprise breaking a seal between the catheter and the needle, in order to facilitate displacement of gas (e.g., air) within the fluidic channel by the flush fluid. Flushing may also remove particulates or other contamination from the fluidic channel and/or catheter. As shown in FIG. 6C, flushing the fluidic channel preferably includes depressing the syringe to deploy the flush fluid throughout the fluidic channel. However, flushing the fluidic channel may include other suitable steps depending on the specific mechanism of the flush fluid source. For instance, flushing the fluidic channel may comprise activating a mechanical pumping system configured to supply a flush fluid to the fluidic channel. Flushing the fluidic channel may include other steps specific to a particular medical protocol, such as flushing for a predetermined length of time, flushing at a predetermined flow rate, and/or flushing a predetermined volume of flush fluid, using, for instance, an embodiment of the system 100 described above. Flushing the fluidic channel may further include inspecting the channel to verify that no gas bubbles exist along the fluidic channel. Inspecting the channel can be performed by a user, or by a sensor configured to detect gas bubbles and integrated into the system 100 described above.

Substantially stopping fluid flow through the fluidic channel at a point, thereby defining a flush fluid volume between the point and the distal end of the catheter S450 functions to help maintain volume of the flush fluid within the fluidic channel. Stopping fluid flow through the fluidic channel equalizes the distribution of pressure (eliminates a pressure differential) to prevent the flush fluid from exiting the fluidic channel. As shown in FIG. 6D, stopping fluid flow through the fluidic channel preferably includes engaging or activating a tubing clamp at a point distal to the point where the flush fluid source is coupled to the fluidic channel and proximal to the catheter. Alternatively, stopping fluid flow through the fluidic channel may include pinching, activating a valve (e.g. a stop cock), inserting a plug, or otherwise preventing flow through at least one end of the fluidic channel. In yet another alternative embodiment, stopping fluid flow through the fluidic channel may comprise leaving the flush fluid source and the fluidic channel coupled, but stopping fluid flow through the fluidic channel, for example, by stopping motion of a syringe plunger or by deactivating a mechanical pumping mechanism. In these examples, the point at which fluid flow is substantially stopped may be defined as the point at which the flush fluid entered the fluidic channel. Stopping fluid flow through the fluidic channel preferably defines a flush fluid volume between the point and the distal end of the catheter (facilitated by the creation of a fluidic seal formed by the catheter tightly fit around the needle).

Preferably, with regard to substantially stopping fluid flow through the fluidic channel at a point, the flush fluid volume between the point and the distal end of the catheter is a fixed, closed flush fluid volume in that on one end of the volume, fluid flow is prevented from passing the point of the fluidic channel and on an opposite end of the volume, fluid flow is prevented from passing a fluidic seal formed between the distal end of the catheter and the needle. In an alternative embodiment, the flush fluid volume between the point and the distal end of the catheter is a fixed, semi-closed flush fluid volume in that on one end of the volume, fluid flow is prevented from passing the point of the fluidic channel and on an opposite end of the volume, fluid flow is prevented from passing the distal end of the catheter by a lack of pressure differential (a result of substantially stopping fluid flow through the fluidic channel).

Shown in FIG. 6E, decoupling the fluidic channel and flush fluid source S460 functions to free the luer or other connector coupled to the fluidic channel. Decoupling the fluidic channel and flush fluid source S460 may further comprise coupling the fluidic channel to another fluid source S465 (e.g., a fluid source comprising medication or intravenous fluids) before, during, or after the catheter has been inserted in the patient. An alternative variation of the method may omit coupling the fluidic channel to another fluid source S465, such as if the flush fluid is identical to the fluid to be administered to the patient.

Inserting the catheter, engaged with the needle, into the patient at an insertion site S470 may include any suitable steps for inserting a needle intravenously or into another conduit. For example, inserting the catheter, engaged with the needle, into the patient may include locating a vein, sterilizing an area around the targeted insertion site, applying a tourniquet proximal to the insertion site, angling the needle relative to the skin surface, and piercing the skin surface at the insertion site. This procedure is well known and understood by one ordinarily skilled in the art; however, inserting the needle may include any suitable step. In a preferred embodiment, as shown in FIG. 6F, inserting the catheter, engaged with the needle into the patient, includes folding the catheter hub and stabilization hub of an embodiment of the system 100 described above towards one another to form the frame of the integrated vascular delivery system into a folded configuration, thereby exposing the needle and catheter outside of the frame. The needle is preferably inserted into the patient after flushing the fluidic conduit, but may alternatively be inserted at any suitable time. Inserting the catheter at the insertion site preferably further includes threading the catheter over the needle in the patient at the insertion site, which may include steps known to one ordinarily skilled in the art. Such a method is known as “over the needle” catheter insertion. An example schematic of inserting a catheter is shown in FIG. 6H (some portions of the integrated vascular delivery system not shown).

The step of allowing a flash fluid to flow to the flash chamber by a fluid path defined by the needle S480 functions to enable the user to verify proper placement of the needle in the blood vessel or other desired conduit or location. As shown in FIGS. 6F and 6G, the flash flows directly from the patient through the fluid path defined by the needle (e.g. a lumen or other fluid path traversing the needle) to the flash chamber, along a volume separate and isolated from the fluidic channel. The vent of the flash chamber exposes the flash chamber to approximately atmospheric pressure or other suitable pressure to provide a pressure differential that allows flash to flow from the inserted needle tip to the flash chamber. The flash return in the flash chamber is preferably visible to the user through any intervening structures (e.g., housing, sheath, slider), but may alternatively be indicated by usage of a sensor (e.g. chemical sensor, impedance sensor) in any suitable manner.

Following insertion of the catheter, the method may further include one or more of the steps described in International Application Number PCT/US11/37230. For example, the method may include one or more of the following steps: pulling the housing of the safety needle away from the catheter hub after catheter insertion, thereby substantially simultaneously withdrawing the needle from the catheter hub and drawing the sheath into an extended position that covers the withdrawn needle; allowing the sheath to lock in the extended position; unfolding the frame such that the frame surrounds the insertion site in an unfolded configuration; securing the frame to the patient at a plurality of anchoring points distributed around the insertion site, thereby stabilizing the catheter relative to the insertion site; connecting a fluid supply to the fluidic channel; allowing the fluid supply to be delivered through the fluidic channel and catheter to the patient; and applying a dressing over the insertion site and the frame. However, the method may include any suitable steps following insertion of the needle, verification of flash return present in the flash chamber, and insertion of the catheter over the needle.

The FIGURES illustrate the architecture, functionality and operation of possible implementations of methods according to preferred embodiments, example configurations, and variations thereof. In this regard, each block in a flowchart or block diagram may represent a module, segment, or method step, which comprises one or more executable instructions for implementing the specified logical function(s). It should also be noted that, in some alternative implementations, the functions noted in the block can occur out of the order noted in the FIGURES. For example, two blocks shown in succession may, in fact, be executed substantially concurrently, or the blocks may sometimes be executed in the reverse order, depending upon the functionality involved.

As a person skilled in the art will recognize from the previous detailed description and from the figures and claims, modifications and changes can be made to the preferred embodiments of the invention without departing from the scope of this invention defined in the following claims.

We claim:

1. A system for inserting a catheter comprising:
a frame comprising: a catheter hub configured to provide a first anchoring point on a patient and configured to receive a catheter insertable in the patient at an insertion site, a stabilization hub configured to provide a second anchoring point on the patient, and a flexible tubular lateral member defining a lumen, extending between the catheter hub and the stabilization hub;
a fluidic channel configured to fluidically communicate with the catheter and transfer a fluid to the catheter;
a housing comprising a needle mount and a flash chamber; and
a needle having a distal end insertable through the frame and the catheter and a proximal end coupled to the needle mount, wherein the needle is configured to provide a fluid path to the flash chamber.

2. The system of claim 1, wherein the frame further comprises a second lateral member extending between the catheter hub and the stabilization hub.

3. The system of claim 2, wherein the frame is configured to form an ellipsoid perimeter about the insertion site.

4. The system of claim 1, wherein the fluidic channel comprises a turnabout portion configured to angularly displace a direction of fluid flow.

5. The system of claim 4, wherein the turnabout portion is configured to angularly displace a direction of fluid flow by approximately 180 degrees.

6. The system of claim 1, wherein the fluidic channel is configured to pass through the lumen of the flexible tubular lateral member, and at least one of the catheter hub and the stabilization hub.

7. The system of claim 1, further comprising a flow control system configured to regulate flow of the fluid through the fluidic channel.

8. The system of claim 1, further comprising a tubing clamp configured to restrict fluid flow through the fluidic channel at a restriction point, thus resulting in a lack of a pressure differential across the fluidic channel.

9. The system of claim 1, further comprising a flush fluid source configured to couple to the fluidic channel and supply a flush fluid to the catheter;

10. The system of claim 9, wherein the flush fluid source comprises a syringe.

11. The system of claim 9, wherein the syringe is configured to couple to the fluidic channel by an extension tubing.

12. The system of claim 9, wherein the flush fluid source includes a mechanical pump.

13. The system of claim 9, wherein the flush fluid source further comprises a temperature regulator.

14. The system of claim 9, wherein the flush fluid comprises saline.

15. The system of claim 1 wherein the flash chamber comprises a fluid reservoir located proximal to the needle mount, and fluidically coupled to the fluid path provided by the needle.

16. The system of claim 15, wherein the flash chamber comprises a vent, wherein the vent is configured to provide a pressure differential between the flash chamber and a distal end of the needle.

17. The system of claim 15, wherein a flash fluid in the flash chamber can be visualized through the housing.

18. The system of claim 1, wherein the needle has a lumen that is configured to form a fluid path to the flash chamber.

19. The system of claim 1, further comprising:
a sheath telescopically engaged with the housing, wherein the sheath is coupleable to the frame such that removal of the needle from the frame draws the sheath over the needle, thereby transitioning the sheath from a retracted position in which the sheath exposes the distal end of the needle to an extended position wherein the sheath substantially surrounds the distal end of the needle;
a slider configured to telescopically engage with at least one of the sheath and the housing; and
wherein the frame operates in a folded configuration and in an unfolded configuration.

20. The system of claim 19, wherein:
in the folded configuration of the frame, the catheter hub and the stabilization hub each couples to at least one of the housing and the sheath; and
in the unfolded configuration of the frame, the first and second anchoring points are distributed about the insertion site to anchor the frame to the patient, thereby stabilizing the catheter.

21. The system of claim 19, wherein a flash fluid in the flash chamber can be visualized through the sheath and the slider.

22. The system of claim 1, further comprising a sensor configured to indicate when a flash fluid has entered at least one of the flash chamber and the fluid path.

23. The system of claim 1, further comprising a septum, coupled to at least one of the catheter hub and the stabilization hub, configured to prevent fluid leakage from the septum after the septum is penetrated.

24. A method for inserting a catheter into a patient comprising:
telescopically engaging the catheter around a needle in fluidic communication with a flash chamber;
coupling a fluidic channel, which fluidically communicates with the catheter, to a flush fluid source supplying a flush fluid;
flushing the fluidic channel with the flush fluid;
substantially stopping fluid flow through the fluidic channel at a point, thereby defining a flush fluid volume between the point and the distal end of the catheter;
decoupling the fluidic channel and the flush fluid source;
inserting the catheter, engaged with the needle, into the patient at an insertion site; and
allowing a flash fluid to flow to the flash chamber by a fluid path defined by the needle.

25. The method of claim 24, wherein telescopically engaging the catheter around a needle comprises forming a fluidic seal that prevents a fluid flow from entering into an annular space between the catheter and the needle.

26. The method of claim 24, wherein coupling the fluidic channel to a flush fluid source supplying a flush fluid comprises coupling the fluidic channel to a syringe supplying saline.

27. The method of claim 24, wherein coupling the fluidic channel to a flush fluid source supplying a flush fluid comprises preparing the flush fluid to a specified temperature.

28. The method of claim 24, wherein flushing the fluidic channel with the flush fluid comprises depressing a syringe configured to deploy the flush fluid throughout the fluidic channel.

29. The method of claim 24, wherein substantially stopping fluid flow through the fluidic channel at a point comprises engaging a tubing clamp at the point.

30. The method of claim 24, wherein inserting the catheter, engaged with the needle, into the patient at an insertion site comprises threading the catheter over the needle, thus disengaging the catheter from the needle.

31. The method of claim 24, wherein allowing a flash fluid to flow to the flash chamber further comprises indicating that the flash fluid has entered at least one of the fluid path and the flash chamber.

32. The method of claim 24, further comprising folding the frame prior to inserting the catheter, and unfolding the frame after allowing a flash fluid to flow to the flash chamber.

33. A system for inserting a catheter comprising:
a frame comprising: a catheter hub configured to provide a first anchoring point on a patient and configured to receive a catheter insertable in the patient at an insertion site, a stabilization hub configured to provide a second anchoring point on the patient, and a flexible tubular lateral member defining a lumen, extending between the catheter hub and the stabilization hub;
a fluidic channel configured to fluidically communicate with the catheter and transfer a fluid to the catheter, wherein the fluidic channel is configured to pass through the lumen of the flexible tubular lateral member and at least one of the catheter hub and the stabilization hub, and wherein the fluidic channel comprises a turnabout portion configured to angularly displace a direction of fluid flow;
an extension tubing configured to couple to the fluidic channel, wherein the extension tubing comprises a tubing clamp configured to restrict the extension tubing at a restriction point thus resulting in a lack of a pressure differential across the fluidic channel; and
a flush fluid source configured to couple to the extension tubing and supply a flush fluid to the catheter.

34. A system for inserting a catheter comprising:
a housing comprising a needle mount and a flash chamber;
a needle having a distal end insertable through the catheter and a proximal end coupled to the needle mount, wherein the needle comprises a lumen configured to provide a fluid path to the flash chamber;
a sheath telescopically engaged with the housing and circumferentially surrounding at least a portion of the needle, wherein the sheath operates in:
a retracted position, wherein the sheath exposes the distal end of the needle, and
an extended position, wherein the sheath substantially surrounds the distal end of the needle,
wherein the sheath is coupleable to a medical device such that removal of the needle from the medical device draws the sheath over the needle, thereby transitioning the sheath from the retracted position to the extended position; and
a slider, longitudinally engaged with at least one of the sheath and the housing and including a restraint that selectively engages the sheath, wherein:
when the restraint is engaged with the sheath, the restraint reinforces the coupling of the sheath to the medical device; and
when the restraint is disengaged from the sheath, the restraint weakens the coupling of the sheath to the medical device.

35. The system of claim 34, wherein a flash fluid in the flash chamber can be visualized through the housing, the sheath, and the slider.
